(19) United States
(12) Patent Application Publication
(10) Pub. No.: US 2019/0382854 A1
(43) Pub. Date: Dec. 19, 2019

(54) GENERIC ASSAYS FOR DETECTION OF INFLUENZA VIRUSES

(71) Applicant: Seqirus UK Limited, Berkshire (GB)

(72) Inventor: Bernhard ROTH, Marburg (DE)

(21) Appl. No.: 16/261,892

(22) Filed: Jan. 30, 2019

Related U.S. Application Data
(63) Continuation of application No. 14/919,593, filed on Oct. 21, 2015, now abandoned.
Application No. 14/919,593 is a continuation of application No. 13/319,333, filed on Apr. 27, 2012, now abandoned.
(60) Provisional application No. 61/217,045, filed on May 26, 2009.
(60) Provisional application No. 61/215,704, filed on May 8, 2009.

(51) Int. Cl. C12Q 1/70; A61K 39/145; C12N 7/00

(52) U.S. Cl. CPC C12Q 1/701; A61K 39/145; C12N 7/00; C12N 2760/16134; C12Q 2600/158; C12N 2760/16161; C12N 2760/16234; C12N 2760/16251; C12N 2760/16261; C12N 2760/16151

(57) ABSTRACT

The invention relates to generic methods for the detection and quantification of influenza viruses. These may uses a reverse transcription (RT-PCR) real time (q-PCR) assay which amplifies a conserved region within influenza A or B strains. The assays allow the quantification of influenza virus RNA molecules or whole virus particles, irrespective of the particular virus strain (e.g. human, avian, swine flu). The methods are particularly applicable as diagnostic assays or in the monitoring of vaccine production processes.

### CROSS REFERENCE TO RELATED APPLICATIONS

This application is a Continuation of U.S. patent application Ser. No. 14/919,593, filed Oct. 21, 2015, which is a Continuation of U.S. patent application Ser. No. 13/319,333, filed Apr. 27, 2012, which is the U.S. National Stage of International Patent Application No. PCT/IB2010/001158, filed May 10, 2010, which claims the benefit of U.S. Provisional Patent Application No. 61/215,704, filed May 8, 2009, and 61/217,045, filed May 26, 2009. The contents of the prior applications are incorporated herein by reference in their entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 223002122501SEQLIST.txt, date recorded: Oct. 21, 2015, size: 4 KB).

### TECHNICAL FIELD

The present invention relates to novel, generic methods for the detection and quantification of influenza viruses. The invention preferably uses a reverse transcription (RT-PCR) Real Time (q-PCR) assay which amplifies a conserved region within influenza A or B strains. The inventive assays allow the quantification of influenza virus RNA molecules or whole virus particles, irrespective of the particular virus strain (e.g. human, avian, swine flu). The inventive methods are particularly applicable as diagnostic assays or in the monitoring of vaccine production processes.

### BACKGROUND OF THE INVENTION

Various forms of influenza vaccines are currently available. Vaccines are generally based either on live virus or on inactivated virus. Inactivated vaccines may be based on whole virions, ‘split’ virions, or on purified surface antigens (see details in WO 2008/068631 to which is expressly referred). Influenza vaccines are typically trivalent and contain two influenza A strains and one influenza B strain. Besides the traditional egg-based production methods for influenza vaccines, different cell culture based manufacturing methods have been described more recently (e.g. see chapters 17 and 18 in: Vaccines, eds. Plotkin & Orenstein; 4th edition, 2004, ISBN: 0-7216-9688-0; Wilschut; Mc Elhaney, Palache in “Influenza”; 2. Edition; Elsevier 2006; ISBN 0-7234-3433-6 Chapter 9).

The application of nucleic acid based detection methods within the influenza vaccine production process (e.g. for quality control processes) has so far been limited. This is due to the fact that the influenza strains used in vaccine production change from season to season, and that thus for every season a new, strain specific detection assay would need to be developed. The present invention provides a novel nucleic acid assay analyzing a conserved region within the genome of influenza A or influenza B strains (irrespective of origin, e.g. human, avian, swine flu). These assays are therefore suitable for analyzing a variety of influenza strains.

### DISCLOSURE OF THE INVENTION

The present invention describes a novel method for detecting influenza virus RNA. The inventive methods analyse a conserved region within the influenza A or influenza B virus genome, preferably the region encoding the matrix (M) protein. The M gene nucleotide sequences from GenBank which were used for an alignment of the influenza A M genes and the influenza B M genes are shown in tables 3 and 4, respectively.

For the analysis, a nucleic acid assay is conducted. A preferred assay is Reverse Transcriptase Polymerase Chain Reaction (RT-PCR). However, equivalent RNA amplification methods are also applicable, as known to the person skilled in the art (Nucleic Acid Sequence Based Amplification or NASBA™ as in U.S. Pat. No. 5,409,818; 3SR™; Transcription Mediated Amplification or TMA™ as in U.S. Pat. No. 5,399,491 etc.). The nucleic acid assay is preferably run as a real time assay (e.g. “qPCR”; Taqman™, Lightcycler™; Scorpion™ etc.).

### DETAILED DESCRIPTION OF THE PREFERRED “ONE STEP RT-REAL TIME PCR”

In a particularly preferred embodiment, a one step RT-real time PCR assay is used (“one step RT-qPCR”). The person skilled in the art is familiar with conducting such “one step RT qPCR” assays. He knows how to find detailed reaction conditions for such amplification. Thus the reverse transcription reaction (RT) and the amplification reaction (qPCR) may be performed in the same vessel (e.g. in a single tube or vial) rather than in separate vessels.

Preferably, commercially available RT-PCR kits are used, e.g. Qiagen QuantiTect™ Virus kit or Invitrogen Super Script™ III Platinum™ kit. The generated fluorescence signals can be analyzed using the respective real time cycler software, as known in the art.

The inventive nucleic acid assays can be quantified by comparing the generated fluorescence signal with the respective signal of a standard nucleic acid, as known in the art. As such standard, a dilution series of an in vitro transcript (IVT) of the respective virus regions is preferably applied. Suitable IVTs can be generated as required or are commercially available e.g. Panomics™ supplies “Ifn-A” (282 nucleotides) and “Ifn-B” (276 nucleotides) single-stranded RNA molecules at 10 ng/ml.

Preferably, RT-q PCR is performed using the primer and probe sequences shown in table 1 below. However, the person skilled in the art knows how to design additional, equivalent primers and probes directed to the virus genome encoding the M protein or to other conserved regions within the influenza genome. The person skilled in the art knows that the Taqman probes shown in the table below can be substituted by equivalent Lightcycler probes or other real time probe systems.

In a particular preferred embodiment, the primers of SEQ ID NO 4 and SEQ ID NO 7 are combined with the probe of SEQ ID NO 3 for the detection of Influenza virus A. In another preferred embodiment, the primers of SEQ ID NO 11 and SEQ ID NO 1 are combined with the probe of SEQ ID NO 9 for the detection of Influenza B viruses.

The examples (see below) show that the inventive one step RT qPCR assay is capable of detecting influenza viruses from different origins.

### Preferred Embodiment: Detection of Intact Viruses

In a particular preferred embodiment, the inventive assays are used to determine the amount of intact virus particles within a sample. This is particularly useful for monitoring vaccine production processes (see in detail below). A differentiation between free virus RNA or nucleoprotein-associated RNA and RNA within virus particles can be achieved by removing the free RNA from the sample prior to the amplification. This can be done, for example, by RNase treatment, as known in the art. A preferred process is as follows:
(a) take a sample of virions, viral RNA and nucleoprotein (e.g. 21 μl volume).
(b) incubate with RNase. For example, use a mixture of RNase A/T1 (15 U/7.5 U) for 1 hour).
(c) dilute the sample. For example, predilutions (1:100-1:10000) of sample are performed using a pipetting robot to get reliable quantitative results in the linear range of the qPCR (Cₜ 15-33);
(d) extract nucleic acid. For example, fully automated vRNA extraction by using magnetic beads;
(e) qPCR. qPCR can be set up by a pipetting robot. The calculation of copies/mL in the sample is in relation to a standard curve of UV-quantified in vitro transcribed RNA (IVT).

Thus the invention provides a method for quantifying the amount of intact virus particles in a sample, comprising steps of: (a) removing non-virion-encapsulated RNA from the sample (e.g. by RNase digestion); (b) amplifying and quantifying remaining RNA in the sample (e.g. as disclosed herein); (c) using the results of step (b) to calculate the amount of intact virus particles in the sample. This method is particularly useful for influenza A and B viruses, especially during vaccine manufacture.

Step (b) may involve quantitative PCR (e.g. RT-PCR). The results of step (b) may be compared to the signal generated by an standard RNA as part of the step (c) calculation. Between steps (a) and (b), virions may be treated to release their RNA, or this release may occur inherently as the PCR process is performed.

Preferred Application of the Invention in Influenza Vaccine Production

The inventive assays are particularly useful for egg-based or cell-culture based influenza vaccine production (for review see: Wilschut; Mc Elhaney, Palache in “Influenza”; 2. Edition; Elsevier 2006; ISBN 0-7234-3433-6 Chapter 9). The invention can be used at different steps during vaccine production, in particular in order to monitor and quantify virus yields in early process stages. The inventive process is in principle suitable for the production of various forms of influenza vaccines (e.g. live virus, inactivated whole virions, ‘split’ virions, purified surface antigens; for details see WO 2008/068631 to which applicant expressly refers). In these production methods, virions are grown in and harvested from virus containing fluids, e.g. allantoic fluid or cell culture supernatant. For the purification of the virions, different methods are applicable, e.g. zonal centrifugation using a linear sucrose gradient solution that includes detergent to disrupt the virions. Antigens may then be purified, after optional dilution, by diafiltration. Split virions are obtained by treating purified virions with detergents (e.g. ethyl-ether, polysorbate 80, deoxycholate, tri-N-butyl phosphate, Triton X-100, Triton N-101, cetyltrimethylammonium bromide, Tergitol NP9, etc.) to produce subvirion preparations, including the ‘Tween-ether’ splitting process. Methods of splitting influenza viruses, for example, are well known in the art (for review see WO 2008/068631). Examples of split influenza vaccines are the BEGRIVAC™, FLUARIX™, FLUZONE™ and FLUSHIELD™ products. The methods of the invention may also be used in the production of live vaccines. The viruses in these vaccines may be attenuated. Live virus vaccines include MedImmune's FLUMIST™ product (trivalent live virus vaccine). Purified influenza virus surface antigen vaccines comprise the surface antigens hemagglutinin and, typically, also neuraminidase. Processes for preparing these proteins in purified form are well known in the art. The FLUVIRIN™, AGRIPPAL™ and INFLUVAC™ products are influenza subunit vaccines. Another form of inactivated antigen is the virosome (nucleic acid free viral like liposomal particles). Virosomes can be prepared by solubilization of virus with a detergent followed by removal of the nucleocapsid and reconstitution of the membrane containing the viral glycoproteins. An alternative method for preparing virosomes involves adding viral membrane glycoproteins to excess amounts of phospholipids to give liposomes with viral proteins in their membrane.

Particularly Preferred Application of the Invention in Cell Culture Based Influenza Vaccine Production

In a particularly preferred embodiment, the invention is used in cell-culture based influenza vaccine production. Suitable cell lines are described e.g. in WO 2008/068631. The most preferred cell lines for growing influenza viruses are MDCK cell lines. The original MDCK cell line is available from the ATCC as CCL-34, but derivatives of this cell line and other MDCK cell lines may also be used. For instance, in WO97/37000 a MDCK cell line is disclosed that was adapted for growth in suspension culture (‘MDCK 33016’, deposited as DSM ACC 2219). Similarly, WO01/64846 discloses a MDCK-derived cell line that grows in suspension in serum-free culture (‘B-702’, deposited as FERM BP-7449). WO2006/071563 discloses non-tumorigenic MDCK cells, including ‘MDCK-S’ (ATCC PTA-6500), ‘MDCK-SFlOl’ (ATCC PTA-6501), ‘MDCK-SF102’ (ATCC PTA-6502) and ‘MDCK-SF103’ (PTA-6503). WO2005/113758 discloses MDCK cell lines with high susceptibility to infection, including ‘MDCK.5F1’ cells (ATCC CRL-12042). Any of these MDCK cell lines can be used.

The cell culture based vaccine production process usually comprises the following steps: The starting material for each monovalent bulk is a single vial of the MDCK working cell bank (WCB). The cells are propagated in a chemically defined medium to optimize cell growth during production. The WCB are expanded by sequential passage in spinner flasks followed by scale up in larger fermentation vessels. Seed virus is added and virus propagation in the fermenter is performed over a period of two to four days. At the end of the infection cycle, the virus suspension is centrifuged and filtered to remove residual intact cells from the culture harvest. The centrifuged, filtered bulk termed clarified virus harvest is the end of the fermentation process. The clarified virus harvest may be stored at room temperature (16-25° C.) in a stainless steel storage vessel for up to 24 hours. The influenza virus is purified by chromatography and ultra-/diafiltration steps, inactivated by beta-propiolactone (BPL) and disrupted by cetyltrimethylammonium bromide (CTAB) to solubilize the viral surface antigens HA and NA. The drug substance production process concludes with a filtration of the concentrate into the final bulk vessel to obtain monovalent bulk. Finally, the monovalent bulks can be blended into multivalent bulks (typically trivalent bulks) and filled into their final container, e.g. syringes. It is standard practice to minimize the amount of residual cell line DNA in the final vaccine, in order to minimize any oncogenic activity of the DNA (see in detail WO 2008/068631).

The present invention can be applied at different points within this process. However, it is particularly preferred that the methods of the invention are used for the monitoring and/or quantifying of virus yields in the early process stages (fermentation, harvest, until inactivation). The inventive assays can be applied as supplemental or alternative methods to the state of the art method (Single radial diffusion (SRD)). The inventive method is rapid (results within ˜4 hours; SRD ˜3 days) and allows an application in high sample throughput. The method is independent from specific reagents (e.g. strain-specific antibodies). The inventive assay is particularly applicable where the sensitivity of SRD is too low (before purification/concentration) or SRD results are unreliable (at harvest/not virus particle associated HA is measured).

In a particularly preferred embodiment, the invention is used to quantify the virus load during the fermentation, in order to determine the optimal time for harvesting the viruses.

In a further particularly preferred embodiment, the nucleic acid analysis is preceded by a RNase digestion of the virus sample. It is such possible to distinguish between free virus RNA and intact virus particles, and thus to quantify the intact virus particles.

Vaccine Compositions and Vaccine Administration

The present invention also provides vaccines produced by the inventive manufacturing processes described above. Such vaccines typically contain HA as the main immunogen, and vaccine doses are standardised by reference to HA levels, typically measured by SRD. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used, as have higher doses (e.g. 3× or 9× doses). Thus vaccines may include between 0.1 and 150 μg of HA per influenza strain, preferably between 0.1 and 50 μg e.g. 0.1-20 μg, 0.1-15 μg, 0.1-10 μg, 0.5-5 μg, etc. Particular doses include e.g. about 45, about 30, about 15, about 10, about 7.5, about 5, about 3.8, about 3.75, about 1.9, about 1.5, etc. per strain. For live vaccines, dosing is measured by median tissue culture infectious dose (TCID50) rather than HA content, and a TCID50 of between 10⁶ and 10⁸ (preferably between 10^{6.5}-10^{7.5}) per strain is typical.

Influenza strains produced with the invention may have a natural HA as found in wild-type viruses, or a modified HA. For instance, it is known to modify HA to remove determinants (e.g. hyper-basic regions around the HA1/HA2 cleavage site) that cause a virus to be highly pathogenic in avian species. The use of so called “reverse genetics” facilitates such modifications.

Influenza virus strains for use in vaccines change from season to season. In interpandemic periods, vaccines typically include two influenza A strains (H1N1 and H3N2) and one influenza B strain, and trivalent vaccines are typical. The invention may also be used in vaccine production against pandemic viral strains (i.e. strains to which the vaccine recipient and the general human population are immunologically naive, in particular of influenza A virus), such as H2, H5, H7 or H9 subtype strains and also H1 subtype strains, and influenza vaccines for pandemic strains may be monovalent or may be based on a normal trivalent vaccine supplemented by a pandemic strain. Depending on the season and on the nature of the antigen included in the vaccine, however, the invention may also be used in vaccine production against one or more of HA subtypes H1, H2, H3, H4, H5, H6, H7, H8, H9, H10, H11, H12, H13, H14, H15 or H16, and/or NA subtypes N1, N2, N3, N4, N5, N6, N7, N8 or N9.

As well as being suitable for the production of vaccines against interpandemic strains, the compositions of the invention are particularly useful for the production of vaccines against pandemic strains. The characteristics of an influenza strain that give it the potential to cause a pandemic outbreak are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naive to the strain's hemagglutinin; (b) it is capable of being transmitted horizontally in the human population; and (c) it is pathogenic to humans. A virus with H5 hemagglutinin type is preferred for immunizing against pandemic influenza, such as a H5N1 strain. Other possible strains include H5N3, H9N2, H2N2, H7N1 and H7N7, and any other emerging potentially pandemic strains and also H1N1 strains.

Compositions of the invention may include antigen(s) from one or more (e.g. 1, 2, 3, 4 or more) influenza virus strains, including influenza A virus and/or influenza B virus. Where a vaccine includes more than one strain of influenza, the different strains are typically grown separately and are mixed after the viruses have been harvested and antigens have been prepared. Thus a process of the invention may include the step of mixing antigens from more than one influenza strain. A trivalent vaccine is typical, including antigens from two influenza A virus strains and one influenza B virus strain. A tetravalent vaccine might also useful, including antigens from two influenza A virus strains and two influenza B virus strains, or three influenza A virus strains and one influenza B virus strain (WO2008/068631).

Vaccine compositions manufactured according to the invention are pharmaceutically acceptable. They usually include components in addition to the antigens e.g. they typically include one or more pharmaceutical carrier(s) and/or excipient(s). As described below, adjuvants may also be included. A thorough discussion of such components is available in reference (WO2008/068631 to which expressly is referred to). Compositions of the invention may advantageously include an adjuvant, which can function to enhance the immune responses (humoral and/or cellular) elicited in a patient who receives the composition. Preferred adjuvants comprise oil-in-water emulsions. Various such adjuvants are known, and they typically include at least one oil and at least one surfactant, with the oil(s) and surfactant(s) being biodegradable (metabolisable) and biocompatible. The oil may comprise squalene. The oil droplets in the emulsion are generally less than 5 μm in diameter, and ideally have a sub-micron diameter, with these small sizes being achieved with a microfluidiser to provide stable emulsions. Droplets with a size of less than 220 nm are preferred as they can be subjected to filter sterilization. Potential adjuvants are described in detail in WO2008/068631 (page 14 following; to which expressly is referred to; e.g. MF59™). Suitable containers for compositions of the invention (or kit components) include sterile vials, syringes (e.g. disposable syringes), nasal sprays, etc. Such containers are described in detail in WO2008/068631 (page 31, to which expressly is referred to).

The invention provides a vaccine manufactured according to the invention. These vaccine compositions are suitable for administration to human patients, and the invention provides a method of raising an immune response in a patient, comprising the step of administering a composition of the invention to the patient (described in detail in WO 2008/068631; pages 32ff), to which expressly is referred to).

Sequences and Kits

Part of the invention is also the primer and probe sequences outlined in Table 1 (SEQ ID NOs 1, 2, 3, 4, 5, 6, 7, 8, 9, 10, and 11). SEQ ID NO: 8 includes two degenerate bases and so may be present as four separate and individual oligonucleotide sequences.

For influenza A: forwards primers include SEQ ID NOs: 5, 2 and 7; a reverse primer is SEQ ID NO: 4; and a useful probe is SEQ ID NO: 3. For influenza B: reverse primers include SEQ ID NOs: 8, 10 and 1; a forwards primer is SEQ ID NO: 11; and a useful probe is SEQ ID NO: 9. SEQ ID NO: 6 is a further forwards primer for influenza A. The term ‘forwards’ is used only for convenience and refers to a primer having the same sense as the ATG-containing coding strand for the matrix proteins. As influenza virus has a negative-stranded genome the terms forwards and reverse may be inverted when referring to hybridization to a viral genomic RNA segment.

A further embodiment of the invention is the specific combination of the primers of SEQ ID NOs 4 and 7 with the probe of SEQ ID NO 3 (Influenza A), and the specific combination of the primers of SEQ ID NOs 11 and 1 with the probe of SEQ ID NO 9 (Influenza B), in particular in the form of a kit. The kit might contain further components, e.g. buffers, polymerases and further reaction components for the amplification. A further part of the present invention is the use of said sequences, combinations and kits for the detection of influenza viruses and in particular for the quantification of viruses within vaccine production processes for diagnostic applications.

Further useful primers are SEQ ID NOs: 12, 13, 14 and 15. Further useful probes are SEQ ID NOs: 16 and 17. SEQ ID NOs: 14 and 15 can be used in combination with SEQ ID NO: 16. SEQ ID NO 17 can be used in combination with SEQ ID NOs: 4 and 7.

Probes of the invention (e.g. SEQ ID NOs: 3 and 9) may be labeled e.g. with a 5′ 6-carboxyfluorescein (6FAM) label and/or a 3′ ‘BlackBerry Quencher’ (BBQ) label.

The invention also provides nucleic acids which comprise a nucleotide sequence selected from SEQ ID NOs 1, 2, 3, 4, 5, 6, 7, 8, 9, 10, or 11. These nucleic acids should be single-stranded with a length of less than 80 nucleotides e.g. less than 50 nucleotides, or less than 30 nucleotides. They can be useful as primers and/or probes for detecting influenza viruses. The nucleic acid may have the same 3′ residue as the relevant SEQ ID NO: i.e. it may comprise a sequence 5′-X-Y-3′ where: Y is a sequence selected from SEQ ID NOs 1, 2, 3, 4, 5, 6, 7, 8, 9, 10, and 11; and X is a nucleotide sequence of 1 or more nucleotides. The nucleic acid with sequence 5′-X-Y-3′ can hybridise to an influenza virus matrix nucleic acid.

### BRIEF DESCRIPTION OF THE DRAWINGS

FIG. 1 shows the correlation of qPCR virus particle measurements with known infectious virus kinetics, measured during infection. The x-axis indicates hours post infection. The y-axis shows the number of copies per mL as assessed by qPCR on a logarithmic scale. The figure illustrates that the curves of qPCR correspond to standard infectious virus titre kinetics at low m.o.i. In particular, virus offspring is measurable in supernatants after 16-24 hrs and maximum infectivity is seen after 48 hrs. Each line shows a different experiment.

FIG. 2 shows the correlation of virus particles as assessed by transmission electron microscopy versus the number of virus particles as assessed by qPCR according to the present invention. The x-axis shows the number of TEM virus particle counts per mL and the y-axis shows the number of copies per mL as assessed by qPCR.

### MODES FOR CARRYING OUT THE INVENTION

Detection of Different Influenza Subtypes

Influenza virus strains are obtained from the German national reference center for animal influenza (Frierich Loeffler Institute, Riems). The strains are cultured on MDCK 33016PF cells and supernatants of passage one and two are analysed using the RT-PCR methods of the invention using either a Qiagen QuantiTect™ Virus kit or an Invitrogen Super Script™ III Platinum™ kit. The primers and probes used are those shown in table 1.

When the QuantiTect™ Virus kit is used the following temperature profiles are run:

measurement of fluorescence signal: 60° C.

ramp rate 2° C./sec

RT step: 50° C. for 20 min

Taq activation 95° C. for 5 min

Main run: 45 cycles: 94° C. for 15 sec; 60° C. for 45 sec (2-step process) or
45 cycles: 94° C. for 15 sec; 60° C. for 30 sec; 72° C. for 30 sec (3-step process).

When the Super Script™ III Platinum™ kit is used the following temperature profiles are run:

measurement of fluorescence signal: 60° C.

ramp rate 2° C./sec

RT step: 50° C. for 15 min

Taq activation 95° C. for 2 min

Main run: 45 cycles: 94° C. for 15 sec, 60° C. for 45 sec

The fluorescent signals are analysed using the respective real time software. The signals are quantified by comparing the generated fluorescent signal with the respective signal of a dilution series of an IVT. The results shown in table 2 demonstrate that all of the tested influenza virus subtypes can be identified with the same set of primer and probe. This shows that the methods of the invention are capable of detecting several different influenza subtypes. The inventors also tested the influenza strains shown in table 7.

In order to assess the sensitivity of the methods of the invention, a serial dilution of samples containing either the A/Bayern/7/95 influenza strain or the B/Baden Württemberg/3/06 influenza strain is done. The samples are subjected to qPCR using either the QuantiTect™ Virus kit or Super Script™ III Platinum™ kit in accordance with the methods of the present invention (as described above) or the Cepheid™ Influenza Virus AB Primer and Probe Set following the manufacturer's protocol. The fluorescent signals are analysed using the respective real time software. The signals are quantified by comparing the generated fluorescent signal with the respective signal of a dilution series of an IVT. The results are shown in tables 5 and 6. For the influenza A strain, it is shown that virus particles are still detectable up to a dilution of 1:1000000 when using the methods of the invention wherein the Cepheid kit can detect virus particles only up to a dilution of 1:100000. For the influenza B strain, virus particles are still detectable up to a dilution of 1:1000000 wherein the Cepheid kit can detect virus particles only up to a dilution of 1:10000. The methods of the invention are therefore much more sensitive than the prior art methods.

Quantification of Influenza Virus Particles in Sample

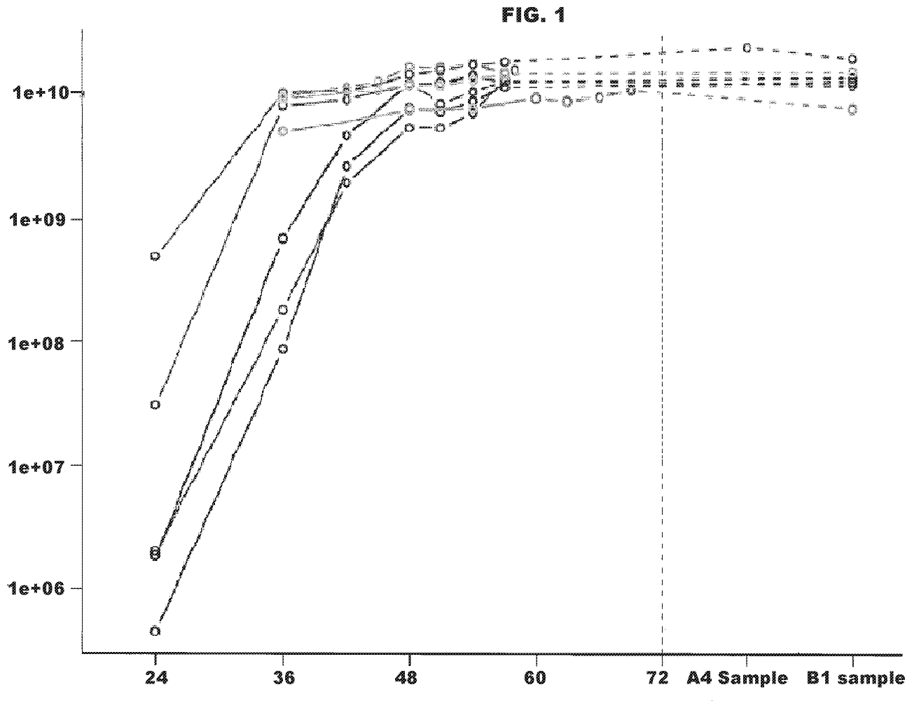

Influenza virus particles are measured in a sample as outlined in FIG. 1. Briefly, a 21 μl sample is subjected to RNase using a mixture of 15 U of RNase A and 7.5 U of T1. The sample is incubated with the RNase mixture for one hour. The pretreated sample is diluted at ratios of 1:100 to 1:10000 in order to obtain a dilution at which qPCR gives results in a linear range. The sample is subjected to qPCR in accordance with the methods of the invention. The results are compared a standard curve using an in vitro transcribed RNA (IVT). The results (illustrated in FIG. 1) show that the obtained curves of qPCR copy numbers correspond to standard infectious virus titre kinetics at low MOI. In particular, virus offspring is measurable in supernatants after 16-24 hours and maximum infectivity titres are observed after 48 hours.

Correlation of Virus Particles as Assessed by Transmission Electron Microscopy and qPCR According to the Present Invention

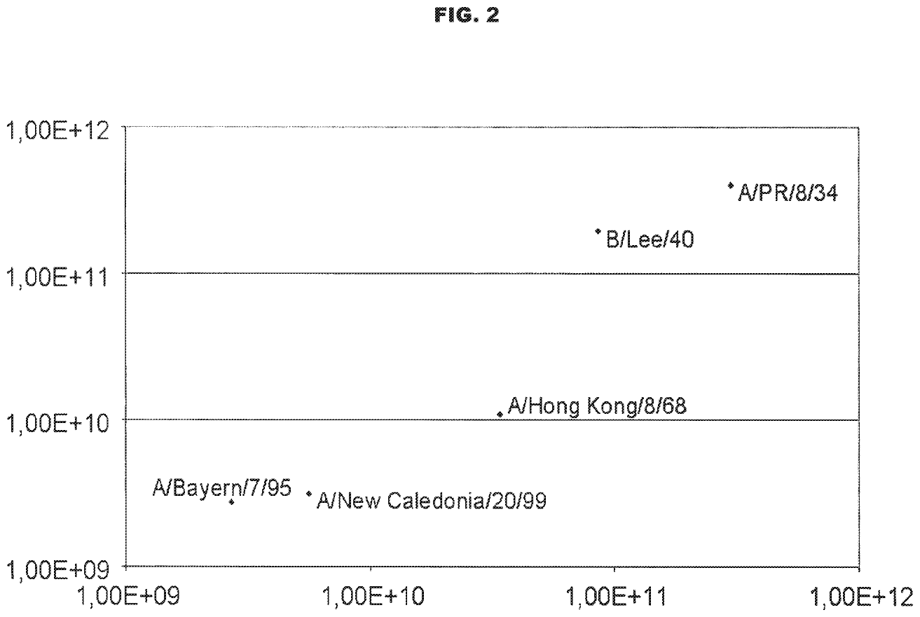

The influenza strains obtained are A/Bayern/7/95, A/New Caledonia/20/99, A/Hong Kong/8/68, B/Lee/40 and A/Puerto Rico/8/34 are obtained from ABI online. The number of virus particles is assessed by transmission electron microscopy (TEM). To this end, virus particles are applied to coated copper grids and air dried. The material is then fixed with 2.5% (v/v) glutaraldehyde, washed and stained with 2% (w/v) aqueous uranyl acetate and 2% (w/v) phosphotungstic acid (PTA) pH 6.5. The number of virus particles in the sample is determined by TEM. The number of viruses particles counted by TEM is compared to the number of virus particles as assessed by qPCR according to the present invention. The results (see FIG. 2) show that the methods of the invention accurately determine the number of virus particles in a sample for various different influenza strains.

It will be understood that the invention has been described by way of example only and modifications may be made whilst remaining within the scope and spirit of the invention.

TABLE 1
preferred primer and probe sequences for the detection of influenza
A and B virus. The probe sequences are Taqman ™ probes.

| SEQ ID | Name | | | |
|---|---|---|---|---|
| NO | (primer/probe) | Sequence | Genus | Tm ° C. |
| 1 | InfBM_BR17 (primer) | gcagatagaggcaccaattagtg | B | 62.9 |
| 2 | InfA_M_BR9 (primer) | caggccccctcaaag | A | 51.6 |
| 3 | InfA_M_TMa (probe) | aggtgacaggattggtcttgtctttagcc | A | 70.4 |
| 4 | InfA_MBR11 (primer) | gcgtctacgctgcagtcc | A | 60.7 |
| 5 | InfA_MBR12 (primer) | cttctaaccgaggtcgaaacg | A | 61.3 |
| 6 | InfA_MBR13 (primer) | gaccaatcctgtcacctctgac | A | 6.0 |
| 7 | InfAM_BR18 (primer) | caggccccctcaaagc | A | 55.8 |
| 8 | InfB_M_BR8 (primer) | gtgctttytgtatatcagttargc | B | 60.1 |
| 9 | InfB_M_TM (probe) | agatggagaaggcaaagcagaactagc | B | 68.3 |
| 10 | InfB_MBR10 (primer) | gatagaggcaccaattagtg | B | 56.4 |
| 11 | InfBM_BR16 (primer) | gtttggagacacaattgcctacc | B | 62.9 |
| 12 | BF (Youil) | ctgtttggagacacaattgc | B | |
| 13 | BR (Youil) | gtgctttytgtatatcag | B | |
| 14 | FluSw H1 F236 | tgggaaatccagagtgtgaatcact | A | |
| 15 | FluSw H1 R318 | cgttccattgtctgaactagrtgtt | A | |
| 16 | FluSw H1 TM292 | ccacaatgtaggaccatgagcttgctgt | A | |
| 17 | InfA_M_swine | aggtgacaagattggtcttgtctttagcc | A | |

TABLE 2

| | | HA-Test | | qPCR copies/ml | |
|---|---|---|---|---|---|
| Sub- | | Passage | | Passage | |
| type | AIV-Strain | 1 | 2 | 1 | 2 |
| H5N6 | A/duck/Potsdam/2243/84 | 128 | 64 | 2.9E+09 | 1.0E+09 |
| H9N2 | A/turkey/Germany/176/ | 128 | 128 | 7.9E+08 | 3.0E+08 |
| | 95 | | | | |
| H3 | R2076/3/07 | 64 | 64 | 6.8E+07 | 3.2E+08 |
| H4N6 | R2619/2/07 | <2 | <2 | 6.1E+02 | <100 |
| H6 | R2707/2/07 | <2 | <2 | 4.9E+03 | <100 |
| H8 | R2709/2/07 | <2 | <2 | 5.1E+02 | <100 |
| H2N1 | R2711/2/07 | <2 | <2 | 2.0E+03 | <100 |

TABLE 3

| | |
|---|---|
| A/Bangkok/1/1979(H3N2) (1027) | A/Wellington/4/2003 (977) |
| A/canine/Texas/1/2004(H3N8) (1026) | A/Canterbury/152/2001 (1000) |
| A/chicken/Bangli Bali/BBPV6-1/2004 (981) | A/Chicken/California/9420/2001(H6N2) (1002) |
| A/chicken/Germany/R28/03(H7N7) (982) | A/Chicken/Shanghai/F/98(H9N2) (1027) |
| A/Dk/ST/5048/2001(H3N8) (988) | A/duck/Hokkaido/9/99 (H9N2) (986) |
| A/Dunedin/10/2002 (977) | A/equine/Ohio/1/2003(H3N8) (1027) |
| A/FPV/Dobson/27 (H7N7) (1027) | A/Hong Kong/1073/99 (1025) |
| A/human/Zhejiang/16/2006(H5N1) (998) | A/Leningrad/134/17/57 (1027) |
| A/mallard/Alberta/24/01 (1027) | A/Memphis/15/1983 (1000) |
| A/New Caledonia/20/1999(H1N1) (985) | A/New York/719/1994 (978) |
| A/seal/Mass/1/80(H7N7) (1027) | A/swan/Mongolia/2/06(H5N1) (987) |
| A/swine/IN/PU542/04 (H3N1) (1030) | A/swine/Iowa/15/1930 (1027) |
| A/Thailand/1(KAN-1)/2004(H5N1) (1039) | A/Thailand/676/2005(H5N1) (1003) |
| A/USSR/90/1977(H1N1) (1027) | A/USSR/90/77 (1000) |
| A/Vietnam/CL119/2005(H5N1) (982) | A/WDk/ST/1737/2000(H6N8) (990) |
| A/WDk/ST/988/2000(H4N9) (815) | |

| | | | | | |
|---|---|---|---|---|---|
| AB166865 | AB188819 | AB189048 | AB212651 | AB239305 | AF046082 |
| AF073180 | AF073181 | AF073182 | AF073185 | AF073186 | AF073187 |
| AF073188 | AF073189 | AF073190 | AF073192 | AF073193 | AF073195 |
| AF073203 | AF073205 | AF156458 | AF156459 | AF156464 | AF156466 |
| AF156467 | AF156468 | AF203788 | AF231360 | AF231361 | AF250486 |
| AF262213 | AF398876 | AF401293 | AF474049 | AF474050 | AF474051 |
| AF474052 | AF474053 | AF474054 | AF474055 | AF474056 | AF474057 |
| AF474058 | AF508687 | AF508692 | AF508693 | AF508694 | AF508695 |
| AF508696 | AF508697 | AF508698 | AF508700 | AF508702 | AY075029 |
| AY130766 | AY210030 | AY210042 | AY210047 | AY210051 | AY210053 |
| AY210054 | AY221537 | AY241600 | AY241612 | AY684709 | AY862620 |
| AY210063 | AY221538 | AY241601 | AY241613 | AY724262 | AY862621 |
| AY210065 | AY241591 | AY241602 | AY241614 | AY724263 | AY862622 |
| AY210244 | AY241592 | AY241603 | AY253755 | AY737292 | AY862623 |
| AY210253 | AY241593 | AY241604 | AY303652 | AY737298 | AY950237 |
| AY210258 | AY241594 | AY241605 | AY303653 | AY770077 | AY950238 |
| AY210264 | AY241595 | AY241606 | AY303654 | AY770998 | AY950239 |
| AY210265 | AY241596 | AY241607 | AY303655 | AY800234 | AY950240 |
| AY221530 | AY241597 | AY241608 | AY303656 | AY818145 | AY950241 |
| AY221531 | AY241598 | AY241609 | AY340091 | AY862615 | CY002955 |
| AY221532 | AY241599 | AY241610 | AY590578 | AY862616 | CY004584 |
| AY221533 | AY651391 | AY241611 | AY609315 | AY862617 | CY004722 |
| AY221536 | AY651413 | CY007036 | AY611525 | CY007220 | CY005445 |
| CY005519 | AY653194 | CY007044 | AY646079 | CY007228 | CY005448 |
| CY005606 | CY006956 | CY007052 | CY007132 | CY007236 | CY007356 |
| CY005653 | CY006964 | CY007060 | CY007140 | CY007244 | CY007364 |
| CY005692 | CY006972 | CY007068 | CY007148 | CY007252 | CY007372 |
| CY005832 | CY006980 | CY007076 | CY007156 | CY007260 | CY007380 |
| CY005839 | CY006988 | CY007084 | CY007164 | CY007268 | CY007388 |
| CY006045 | CY006996 | CY007092 | CY007172 | CY007292 | CY007396 |
| CY006924 | CY007004 | CY007100 | CY007188 | CY007300 | CY007404 |
| CY006932 | CY007012 | CY007108 | CY007196 | CY007316 | CY007412 |
| CY006940 | CY007020 | CY007116 | CY007204 | CY007340 | CY007420 |
| CY006948 | CY007028 | CY007124 | CY007212 | CY007348 | CY007428 |
| CY007436 | CY007668 | CY007868 | CY008084 | CY008349 | CY008589 |
| CY007444 | CY007676 | CY007876 | CY008092 | CY008357 | CY008597 |
| CY007452 | CY007684 | CY007884 | CY008100 | CY008365 | CY008605 |
| CY007460 | CY007692 | CY007892 | CY008108 | CY008373 | CY008613 |
| CY007468 | CY007700 | CY007900 | CY008132 | CY008381 | CY008621 |
| CY007476 | CY007708 | CY007916 | CY008140 | CY008389 | CY008629 |
| CY007484 | CY007716 | CY007924 | CY008157 | CY008397 | CY008637 |
| CY007492 | CY007724 | CY007932 | CY008197 | CY008405 | CY008645 |
| CY007500 | CY007732 | CY007940 | CY008221 | CY008413 | CY008653 |
| CY007508 | CY007740 | CY007948 | CY008229 | CY008421 | CY008749 |
| CY007516 | CY007748 | CY007956 | CY008237 | CY008429 | CY008757 |
| CY007524 | CY007756 | CY007964 | CY008245 | CY008437 | CY008765 |
| CY007532 | CY007764 | CY007988 | CY008253 | CY008445 | CY008773 |
| CY007540 | CY007772 | CY007996 | CY008261 | CY008477 | CY008781 |
| CY007548 | CY007780 | CY008004 | CY008269 | CY008485 | CY008789 |
| CY007556 | CY007788 | CY008012 | CY008277 | CY008493 | CY008797 |
| CY007564 | CY007796 | CY008020 | CY008285 | CY008501 | CY008805 |
| CY007572 | CY007804 | CY008028 | CY008293 | CY008509 | CY008821 |
| CY007580 | CY007812 | CY008036 | CY008301 | CY008541 | CY008829 |
| CY007588 | CY007820 | CY008044 | CY008309 | CY008549 | CY008837 |
| CY007596 | CY007828 | CY008052 | CY008317 | CY008557 | CY008845 |
| CY007604 | CY007844 | CY008060 | CY008325 | CY008565 | CY008853 |
| CY007652 | CY007852 | CY008068 | CY008341 | CY008573 | CY008861 |
| CY007660 | CY007860 | CY008076 | CY008333 | CY008581 | CY009021 |
| CY009029 | CY009581 | CY010141 | CY010413 | CY015054 | DQ064395 |
| CY009037 | CY009589 | CY010149 | CY010421 | CY015074 | DQ064396 |
| CY009045 | CY009597 | CY010157 | CY010429 | CY015082 | DQ064397 |
| CY009077 | CY009621 | CY010165 | CY010437 | CY015097 | DQ064398 |
| CY009085 | CY009757 | CY010173 | CY010445 | CY015109 | DQ064399 |
| CY009093 | CY009765 | CY010181 | CY010453 | CY015116 | DQ064402 |
| CY009101 | CY009789 | CY010189 | CY010461 | CY015152 | DQ064403 |
| CY009109 | CY009797 | CY010197 | CY010469 | CY015444 | DQ064404 |
| CY009117 | CY009805 | CY010205 | CY010477 | CY016125 | DQ064405 |
| CY009133 | CY009813 | CY010213 | CY010549 | CY016277 | DQ064406 |
| CY009141 | CY009821 | CY010221 | CY010557 | CY016285 | DQ064407 |
| CY009149 | CY009829 | CY010229 | CY010765 | CY016293 | DQ083661 |
| CY009157 | CY009845 | CY010237 | CY010773 | CY016301 | DQ083665 |
| CY009165 | CY009853 | CY010245 | CY010781 | DQ009919 | DQ083666 |
| CY009181 | CY009861 | CY010253 | CY011065 | DQ021745 | DQ083668 |
| CY009189 | CY009877 | CY010261 | CY011081 | DQ021746 | DQ083669 |
| CY009197 | CY009885 | CY010269 | CY011089 | DQ021758 | DQ083670 |
| CY009213 | CY009933 | CY010277 | CY011409 | DQ064381 | DQ083671 |
| CY009221 | CY009941 | CY010285 | CY011777 | DQ064382 | DQ083672 |
| CY009229 | CY009949 | CY010293 | CY011785 | DQ064383 | DQ083675 |
| CY009277 | CY009957 | CY010301 | CY012433 | DQ064384 | DQ083678 |
| CY009389 | CY009965 | CY010309 | CY013241 | DQ064385 | DQ083679 |
| CY009397 | CY009973 | CY010317 | CY014672 | DQ064386 | DQ083682 |
| CY009413 | CY009981 | CY010325 | CY014822 | DQ064387 | DQ083686 |
| CY009421 | CY010085 | CY010333 | CY014881 | DQ064388 | DQ083687 |
| CY009437 | CY010093 | CY010341 | CY014910 | DQ064389 | DQ083688 |
| CY009533 | CY010101 | CY010349 | CY015007 | DQ064390 | DQ083690 |
| CY009541 | CY010109 | CY010365 | CY015015 | DQ064391 | DQ083692 |
| CY009549 | CY010117 | CY010381 | CY015020 | DQ064392 | DQ083697 |
| CY009557 | CY010125 | CY010389 | CY015028 | DQ064393 | DQ094252 |
| CY009573 | CY010133 | CY010397 | CY015034 | DQ064394 | DQ094255 |
| DQ094256 | DQ094270 | DQ095642 | DQ320961 | DQ320999 | DQ376656 |
| DQ094257 | DQ095632 | DQ095643 | DQ320964 | DQ321000 | DQ376657 |
| DQ094258 | DQ095633 | DQ095644 | DQ320976 | DQ321003 | DQ376658 |
| DQ094259 | DQ095635 | DQ095645 | DQ320992 | DQ321006 | DQ376659 |
| DQ094260 | DQ095636 | DQ095646 | DQ320993 | DQ323678 | DQ376660 |
| DQ094261 | DQ095637 | DQ236084 | DQ320994 | DQ351858 | DQ376662 |
| DQ094262 | DQ095638 | DQ237951 | DQ320995 | DQ351859 | DQ376664 |
| DQ094263 | DQ095639 | DQ320942 | DQ320996 | DQ351860 | DQ376666 |
| DQ094264 | DQ095640 | DQ320959 | DQ320997 | DQ366333 | DQ376667 |
| DQ094265 | DQ095641 | DQ320960 | DQ320998 | DQ376655 | DQ376668 |
| DQ376669 | DQ376689 | DQ492913 | DQ492939 | DQ492979 | DQ997179 |
| DQ376670 | DQ449633 | DQ492915 | DQ492940 | DQ508828 | DQ997226 |
| DQ376671 | DQ482663 | DQ492916 | DQ492943 | DQ508836 | DQ997304 |
| DQ376672 | DQ482664 | DQ492917 | DQ492948 | DQ643985 | DQ997457 |
| DQ376673 | DQ482665 | DQ492918 | DQ492952 | DQ650660 | DQ997473 |
| DQ376674 | DQ485211 | DQ492920 | DQ492953 | DQ650664 | DQ997488 |
| DQ376675 | DQ485219 | DQ492921 | DQ492954 | DQ676831 | DQ997498 |
| DQ376676 | DQ485227 | DQ492922 | DQ492956 | DQ676835 | DQ997512 |
| DQ376677 | DQ492903 | DQ492923 | DQ492957 | DQ676839 | FLAM1A |
| DQ376678 | DQ492904 | DQ492925 | DQ492959 | DQ849018 | FLAM1M2A |
| DQ376679 | DQ492905 | DQ492926 | DQ492961 | DQ997086 | FLAM2C |
| DQ376680 | DQ492906 | DQ492927 | DQ492964 | DQ997099 | IAU49119 |
| DQ376683 | DQ492907 | DQ492928 | DQ492967 | DQ997108 | IAU65564 |
| DQ376684 | DQ492909 | DQ492929 | DQ492971 | DQ997119 | IAU65571 |
| DQ376686 | DQ492911 | DQ492930 | DQ492973 | DQ997124 | IAU65574 |
| DQ376688 | DQ492912 | DQ492937 | DQ492975 | DQ997138 | |

TABLE 4

| | |
|---|---|
| B/Aichi/5/88 (1142) | B/Ann Arbor/1/1986 (1155) |
| B/Bangkok/460/03 (1074) | B/Barcelona/215/03 (1088) |
| B/Beijing/184/93 (1096) | B/Bucharest/795/03 (1087) |
| B/England/23/04 (1053) | B/Hong Kong/05/1972 (1154) |
| B/Hong Kong/330/2001 (1190) | B/Houston/1/91 (1079) |
| B/Ibaraki/2/85 (1141) | B/Jiangsu/10/03 (1059) |
| B/Lee/40 (1191) | B/Los Angeles/1/02 (1076) |
| B/Memphis/13/03 (1076) | B/Nanchang/6/96 (1076) |
| B/Oslo/71/04 (1095) | B/Panama/45/90(1139) |
| B/Singapore/222/79 (1187) | B/Trento/3/02 (1077) |
| B/Victoria/504/2000 (1190) | B/Yamagata/1/73 (1188) |
| B/Yamagata/K519/2001 (1076) | |

| | | | | | | |
|---|---|---|---|---|---|---|
| AB036878 | AF100375 | AF100377 | AF100380 | AF100381 | AF100382 | AF100383 |
| AF100384 | AF100385 | AF100386 | AF100387 | AF100388 | AJ783377 | AJ783379 |
| AJ783381 | AJ783382 | AJ783383 | AJ783384 | AJ783386 | AJ783387 | AJ783388 |
| AJ783392 | AJ783393 | AJ783394 | AJ783395 | AY260941 | AY260955 | AY504613 |
| AY504621 | AY581971 | AY581982 | AY581983 | AY687399 | DQ508916 | DQ508924 |
| FLBMO | NC_002210 | | | | | |

TABLE 5
A/Bayern/7/95

| | | | | Cₜ | Cₜ |
|---|---|---|---|---|---|
| | | Cₜ | Cₜ | Super- | Cepheid |
| | | Quantitect Virus | | script | Influenza Virus |
| | TCID50/mL | (Field-Test) | | III | A/B Primer and |
| Dilution | (8.1) | 3-step | 2-step | Platinum | Probe Set ASR |
| undiluted | 125.892.541 | 14.26 | 13.05 | 12.43 | 15.76 |
| 1:10 | 12.589.254 | 17.49 | 16.18 | 15.96 | 18.52 |
| 1:100 | 1.258.925 | 20.70 | 19.92 | 20.29 | 22.14 |
| 1:1000 | 125.893 | 24.50 | 22.77 | 23.22 | 25.23 |
| 1:10000 | 12.589 | 27.72 | 26.55 | 26.31 | 28.96 |
| 1:100000 | 1.259 | 31.12 | 30.14 | 29.25 | 33.26 |
| 1:1000000 | 126 | 35.57 | 36.68 | 32.99 | 0.00 |

TABLE 6
B/Baden-Württemberg/3/06

| | | | | Cₜ | Cₜ |
|---|---|---|---|---|---|
| | | Cₜ | Cₜ | Super- | Cepheid |
| | | Quantitect Virus | | script | Influenza Virus |
| | TCID50/mL | (Field-Test) | | III | A/B Primer and |
| Dilution | (7.9) | 3-step | 2-step | Platinum | Probe Set ASR |
| Undiluted | 79.432.823 | 15.47 | 14.49 | 13.59 | 16.91 |
| 1:10 | 7.943.282 | 18.61 | 18.10 | 16.99 | 19.45 |
| 1:100 | 794.328 | 22.20 | 21.32 | 20.96 | 23.29 |
| 1:1000 | 79.433 | 25.28 | 24.27 | 24.66 | 27.10 |
| 1:10000 | 7.943 | 28.92 | 28.11 | 28.16 | 30.96 |
| 1:100000 | 794 | 0.00 | 31.54 | 31.07 | 0.00 |
| 1:1000000 | 79 | 0.00 | 36.96 | 34.77 | 0.00 |
| 1:10000000 | 8 | | | | |

TABLE 7

| | | |
|---|---|---|
| A/Bayem/7/95 | A/New Caledonia/20/99 | B/Brisbane/60/2008 |
| A/Brisbane/59/2007 IVR 148 | A/PR/8/34 | B/BadenWuerttemberg/3/2006 |
| A/Brisbane/10/2007 IVR 147 | A/Solomon Islands/03/2006 | B/Florida/04/2006 |
| A/California/04/2009 | A/Uruguay 716/07 X-175C | B/Fujian/1272/2008 |
| A/California/07/2009 X179A | A/Uruguay/716/07 | B/Lee/40 |
| A/HH/01/2009 | A/Wisconsin/67/2005 | B/Malaysia/2506/2004 |
| A/HK/8/68 | B/Bangladesh/3333/2007 | B/Perth/210/2008 |
| A/Mexiko/4108/2009 | B/Brisbane/33/2008 | |

1.-16. (canceled)

17. A method of providing a doctor with an influenza diagnosis comprising:
a) receiving a request from a doctor comprising a sample,
b) detecting influenza virus RNA in the sample, wherein a conserved region within the influenza genome is amplified, and
c) providing the doctor with an influenza diagnosis.

18. The method of claim 17, wherein the conserved region encodes partly or completely an M protein of the influenza virus.

19. The method of claim 17, wherein the conserved region comprises SEQ ID NO: 3 or SEQ ID NO: 9.

20. The method of claim 17, wherein the conserved region is amplified by a one-step RT-qPCR.

21. The method of claim 20, wherein the one-step RT-qPCR uses at least one primer selected from the group consisting of SEQ ID NOs: 1-11.

22. The method of claim 17, wherein the influenza diagnosis in c) comprises quantifying the amount of intact virus particles in the sample.

23. The method of claim 22, comprising comparing a signal generated for influenza virus RNA in the sample and a signal generated for a standard RNA, thereby quantifying the amount of intact virus particles in the sample.

24. A method of ordering an influenza vaccine, comprising:
a) receiving a sample during a fermentation step of an influenza vaccine production;
b) removing free virus RNA from the sample;
c) recording a first signal of influenza virus RNA in the sample after b);
d) recording a second signal of influenza virus RNA in a control sample that contains a defined amount of influenza virus RNA;
e) comparing the first signal to the second signal, thereby quantifying the amount of intact virus particles in the sample; and
f) updating a user to indicate the amount of intact virus particles in the sample, thereby allowing the user to determine an optimal time for harvesting viruses for influenza vaccine production.

25. The method of claim 24, wherein the recording in step c) and d) comprises amplifying a conserved region within the genome of the influenza virus.

26. The method of claim 25, wherein the conserved region encodes partly or completely an M protein of the influenza virus.

27. The method of claim 25, wherein the conserved region comprises SEQ ID NO: 3 or SEQ ID NO: 9.

28. The method of claim 25, wherein the amplifying comprises performing a one-step RT-qPCR.

29. The method of claim 28, wherein the one-step RT-qPCR uses at least one primer selected from the group consisting of SEQ ID NOs: 1-11.

30. The method of claim 24, wherein the removing in step b) is performed by RNase treatment.

31. The method of claim 24, further comprising:
g) centrifuging and filtering the harvested viruses;
h) purifying the viruses after g) by chromatography and ultra-/diafiltration steps, inactivating the viruses, and disrupting the viruses to solubilize the viral surface antigens HA and NA;
i) filtering the HA and NA antigens to obtain a monovalent bulk; and
j) optionally, blending the monovalent bulk into multivalent bulks and filling into a final container.


SEQUENCE LISTING (17 sequences)

SEQ ID NO: 1 - DNA, 23 bp, Artificial Sequence
  Primer Sequence
gcagatagaggcaccaattagtg

SEQ ID NO: 2 - DNA, 15 bp, Artificial Sequence
  Primer Sequence
caggccccctcaaag

SEQ ID NO: 3 - DNA, 29 bp, Artificial Sequence
  Probe Sequence
aggtgacaggattggtcttgtctttagcc

SEQ ID NO: 4 - DNA, 18 bp, Artificial Sequence
  Primer Sequence
gcgtctacgctgcagtcc

SEQ ID NO: 5 - DNA, 21 bp, Artificial Sequence
  Primer Sequence
cttctaaccgaggtcgaaacg

SEQ ID NO: 6 - DNA, 22 bp, Artificial Sequence
  Primer Sequence
gaccaatcctgtcacctctgac

SEQ ID NO: 7 - DNA, 16 bp, Artificial Sequence
  Primer Sequence
caggccccctcaaagc

SEQ ID NO: 8 - DNA, 24 bp, Artificial Sequence
  Primer Sequence
  misc_feature 8: y = c or t
  misc_feature 22: r = a or g
gtgctttytgtatatcagttargc

SEQ ID NO: 9 - DNA, 27 bp, Artificial Sequence
  Probe Sequence
agatggagaaggcaaagcagaactagc

SEQ ID NO: 10 - DNA, 20 bp, Artificial Sequence
  Primer Sequence
gatagaggcaccaattagtg

SEQ ID NO: 11 - DNA, 23 bp, Artificial Sequence
  Primer Sequence
gtttggagacacaattgcctacc

SEQ ID NO: 12 - DNA, 20 bp, Artificial Sequence
  Primer Sequence
ctgtttggagacacaattgc

SEQ ID NO: 13 - DNA, 18 bp, Artificial Sequence
  Primer Sequence
  misc_feature 8: y = c or t
gtgctttytgtatatcag

SEQ ID NO: 14 - DNA, 25 bp, Artificial Sequence
  Primer Sequence
tgggaaatccagagtgtgaatcact

SEQ ID NO: 15 - DNA, 25 bp, Artificial Sequence
  Primer Sequence
  misc_feature 21: r = a or g
cgttccattgtctgaactagrtgtt

SEQ ID NO: 16 - DNA, 28 bp, Artificial Sequence
  Probe Sequence
ccacaatgtaggaccatgagcttgctgt

SEQ ID NO: 17 - DNA, 29 bp, Artificial Sequence
  Primer Sequence
aggtgacaagattggtcttgtctttagcc